(19) United States
(12) Patent Application Publication
(10) Pub. No.: US 2009/0076342 A1
(43) Pub. Date: Mar. 19, 2009

(54) Adherent Multi-Sensor Device with Empathic Monitoring

(75) Inventors: Badri Amurthur, Los Gatos, CA (US); Mark J. Bly, Falcon Heights, MN (US); Kristofer J. James, Eagan, MN (US); Imad Libbus, Saint Paul, MN (US); Yatheendhar D. Manicka, Woodbury, MN (US); Scott T. Mazar, Woodbury, MN (US); Jerry S. Wang, Blaine, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Corventis, Inc., San Jose, CA (US)

(21) Appl. No.: 12/209,271

(22) Filed: Sep. 12, 2008

Related U.S. Application Data
(60) Provisional application No. 60/972,537, filed on Sep. 14, 2007.
(60) Provisional application No. 60/972,359, filed on Sep. 14, 2007.
(60) Provisional application No. 60/972,336, filed on Sep. 14, 2007.
(60) Provisional application No. 60/972,340, filed on Sep. 14, 2007.
(60) Provisional application No. 61/055,666, filed on May 23, 2008.

(51) Int. Cl. A61B 5/00

(52) U.S. Cl. 600/301

(57) ABSTRACT

A system and methods are provided for physiological monitoring and prediction. The system includes a detecting system and a remote monitoring system. The detecting system includes, (i) an adherent device with a plurality of sensors that provide an indication of at least one physiological event of a patient, the adherent device being coupled to the patient's thorax and (ii) a wireless communication device coupled to the plurality of sensors and configured to transfer patient data directly or indirectly from the plurality of sensors to a remote monitoring system. The remote monitoring system is coupled to the wireless communication device. Logic resources may be provided at the remote monitoring system to determine the physiological event of the patient.

### CROSS-REFERENCES TO RELATED APPLICATIONS

The present application claims the benefit under 35 USC 119(e) of U.S. Provisional Application Nos. 60/972,359, 60/972,537, 60/972,340 and 60/972,336 all of which were filed on Sep. 14, 2007, and 61/055,666 filed May 23, 2007; the full disclosures of which are incorporated herein by reference in their entirety.

The subject matter of the present application is related to the following applications: 60/972,512; 60/972,329; 60/972,354; 60/972,616; 60/972,363; 60/972,343; 60/972,581; 60/972,629; 60/972,316; 60/972,333 all of which were filed on Sep. 14, 2007; 61/046,196 filed Apr. 18, 2008; 61/047,875 filed Apr. 25, 2008; 61/055,645, 61/055,656, 61/055,662 all filed May 23, 2008; and 61/079,746 filed Jul. 10, 2008.

The following applications are being filed concurrently with the present application, on Sep. 12, 2008: Attorney Docket Nos. 026843-000110US entitled “Multi-Sensor Patient Monitor to Detect Impending Cardiac Decompensation Prediction”; 026843-000220US entitled “Adherent Device with Multiple Physiological Sensors”; 026843-000410US entitled “Injectable Device for Physiological Monitoring”; 026843-000510US entitled “Delivery System for Injectable Physiological Monitoring System”; 026843-000620US entitled “Adherent Device for Cardiac Rhythm Management”; 026843-000710US entitled “Adherent Device for Respiratory Monitoring”; 026843-000810US entitled “Adherent Athletic Monitor”; 026843-000910US entitled “Adherent Emergency Monitor”; 026843-001320US entitled “Adherent Device with Physiological Sensors”; 026843-001410US entitled “Medical Device Automatic Start-up upon Contact to Patient Tissue”; 026843-001900US entitled “System and Methods for Wireless Body Fluid Monitoring”; 026843-002010US entitled “Adherent Cardiac Monitor with Advanced Sensing Capabilities”; 026843-002410US entitled “Adherent Device for Sleep Disordered Breathing”; 026843-002710US entitled “Dynamic Pairing of Patients to Data Collection Gateways”; 026843-003010US entitled “Adherent Multi-Sensor Device with Implantable Device Communications Capabilities”; 026843-003110US entitled “Data Collection in a Multi-Sensor Patient Monitor”; 026843-003310US entitled “Energy Management for Adherent Patient Monitor”; and 026843-00341US entitled “Tracking and Security for Adherent Patient Monitor.”

### BACKGROUND OF THE INVENTION

1. Field of the Invention

This invention relates generally to systems and methods for remote monitoring of a patient.

Frequent monitoring of patients permits the patients' physician to detect worsening symptoms as they begin to occur, rather than waiting until a critical condition has been reached. As such, home monitoring of patients with chronic conditions is becoming increasingly popular in the health care industry for the array of benefits it has the potential to provide. Potential benefits of home monitoring are numerous and include: better tracking and management of chronic disease conditions, earlier detection of changes in the patient condition, and reduction of overall health care expenses associated with long term disease management. The home monitoring of a number of diverse “chronic diseases” is of interest, where such diseases include diabetes, dietary disorders such as anorexia and obesity, anxiety, depression, epilepsy, respiratory diseases, AIDS and other chronic viral conditions, conditions associated with the long term use of immunosuppressants, e.g. in transplant patients, asthma, chronic hypertension, chronic use of anticoagulants, and the like.

Of particular interest in the home monitoring sector of the health care industry is the remote monitoring of patients with heart failure (hereinafter “HF”), also known as congestive heart failure. HF is a syndrome in which the heart is unable to efficiently pump blood to the vital organs. Most instances of HF occur because of a decreased myocardial capacity to contract (systolic dysfunction). However, HF can also result when an increased pressure-stroke-volume load is imposed on the heart, such as when the heart is unable to expand sufficiently during diastole to accommodate the ventricular volume, causing an increased pressure load (diasystolic dysfunction).

In either case, HF is characterized by diminished cardiac output and/or damming back of blood in the venous system. In HF, there is a shift in the cardiac function curve and an increase in blood volume caused in part by fluid retention by the kidneys. Indeed, many of the significant morphologic changes encountered in HF are distant from the heart and are produced by the hypoxic and congestive effects of the failing circulation upon other organs and tissues. One of the major symptoms of HF is edema, which has been defined as the excessive accumulation of interstitial fluid, either localized or generalized.

HF is the most common indication for hospitalization among adults over 65 years of age, and the rate of admission for this condition has increased progressively over the past two decades. It has been estimated that HF affects more than 3 million patients in the U.S. (J. B. O'Connell et al., J. Heart Lung Transpl. (1993) 13(4):S107-112).

In the conventional management of HF patients, where help is sought only in crisis, a cycle occurs where patients fail to recognize early symptoms and do not seek timely help from their care-givers, leading to emergency department admissions (Miller, P. Z., 1995, “Home monitoring for congestive heart failure patients,” Caring Magazine, August 1995: 53-54). Recently, a prospective, randomized trial of 282 patients was conducted to assess the effect of the intervention on the rate of admission, quality of life, and cost of medical care. In this study, a nurse-directed, multi disciplinary intervention (which consisted of comprehensive education of the patient and family, diet, social-service consultation and planning, review of medications, and intensive assessment of patient condition and follow-up) resulted in fewer readmissions than the conventional treatment group and a concomitant overall decrease in the cost of care (M. W. Rich et al., New Engl. J. Med. (1995) 333:1190-95).

Similarly, comprehensive discharge planning and a home follow-up program was shown to decrease the number of readmissions and total hospital charges in an elderly population (M. Naylor et al., Amer. College Physicians (1994) 120:999-1006). Therefore, home monitoring is of particular interest in the HF management segment of the health care industry.

Another area in which home-monitoring is of particular interest is in the remote monitoring of a patient parameter that provides information on the titration of a drug, particularly with drugs that have a consequential effect following administration, such as insulin, anticoagulants, ACE inhibitors, .beta.-blockers, diuretics, etc.

Although a number of different home monitoring systems have been developed, there is continued interest in the development of new monitoring systems. Of particular interest would be the development of a system that provides for improved patient compliance, ease of use, etc. Of more particular interest would be the development of such a system that is particularly suited for use in the remote monitoring of patients suffering from HF.

There is a need for an improved home monitoring of patients with chronic conditions. There is a further need for an improved HF monitoring system.

2. Description of the Background Art

The following U.S. patents and Publications may describe relevant background art: U.S. Pat. Nos. 4,121,573; 4,955,381; 4,981,139; 5,080,099; 5,353,793; 5,511,553; 5,544,661; 5,558,638; 5,724,025; 5,772,586; 5,862,802; 6,047,203; 6,117,077; 6,129,744; 6,225,901; 6,385,473; 6,416,471; 6,454,707; 6,527,711; 6,527,711; 6,527,729; 6,551,252; 6,595,927; 6,595,929; 6,595,929; 6,605,038; 6,645,153; 6,821,249; 6,980,851; 7,020,508; 7,054,679; 7,153,262; 2003/0092975; 2005/0113703; 2005/0131288; 2006/0010090; 2006/0031102; 2006/0089679; 2006/0122474; 2006/0155183; 2006/0224051; 2006/0264730; 2007/0021678; and 2007/0038038.

### SUMMARY OF THE INVENTION

Accordingly, embodiments of the present invention provide an improved systems, methods and devices for monitoring patients, for example patients with chronic conditions such as HF, and for detecting a physiologic event of the patient. The system may comprise a patient measuring system, also referred to as a patient detection system, and a remote monitoring system. The patient measuring system may comprise an adherent device configured to couple to the patient, and a wireless communication device, such as a gateway. Circuitry can be configured to receive the patient data and configured to determine, for example to detect, a physiological event of the patient. In response to the detected physiological event, the remote center can contact, for example solicit, the patient to determine how he or she is feeling and what she is thinking, so as to gather empathic information of the patient. This subjective empathic information can be combined with data from the patient measuring system to better assess the patient and may be used to send a notification to a physician, nurse or care provider, such that the accuracy of the notification is confirmed based on the subjective empathic information from the patient. At least one of the patient measuring system or the remote monitoring system may comprise the circuitry, and the circuitry may comprise at least one of a processor comprising a tangible medium, a processor system or discrete logic circuitry. For example, the remote monitoring system may comprise the circuitry, and the circuitry may be located remotely from the patient. In many embodiments, the remote monitoring system uses outputs of a plurality of sensors having multiple features to enhance physiological sensing performance, for example by combining the output of two or more sensors. For example, heart failure status can be determined by a weighted combination of sensor output measurement signals, for example a weighted combination of a change in sensor output from a baseline value.

In a first aspect, embodiments of the present invention provide a system for detecting at least one physiological event of a patient. The system comprises a patient measuring system and remote monitoring system. The patient measuring system comprises an adherent device configured to couple to the patient. The adherent device comprises a plurality of sensors to measure patient data for detecting at least one physiological event of the patient. A wireless communication device is coupled to the plurality of sensors and configured to transmit wirelessly the patient data. The remote monitoring system is coupled to the wireless communication device and configured to receive the patient data. Circuitry, also referred to as logic resources, is configured to receive the patient data and detect the physiological event of the patient.

In many embodiments, the remote monitoring system is configured to contact the patient to determine at least one of patient feelings or patient thoughts in response to the circuitry the physiologic event of the patient. The remote site may be configured to combine the at least one of the patient feeling or the patient thought with the patient data from the adherent device and send a notification in response to the patient data combined with the at least one of the patient feeling or the patient thought.

In many embodiments, the plurality of sensors comprises a combination of sensors to measure at least two of an electrocardiogram signal, a hydration signal, an accelerometer signal or a respiration signal of the patient.

In many embodiments, the patient measuring system further comprises an external device coupled to patient to measure patient data in combination with the adherent device. The external device may comprise at least one of a weight scale, a blood pressure cuff, a medical treatment device or a medicament dispenser.

In many embodiments, the system comprises a monitoring unit, and at least one of the patient measuring system or the remote monitoring system comprises the monitoring unit. The external device and the adherent device may be coupled to the monitoring unit, and the remote monitoring system may comprise monitoring unit. The external device and the adherent device can be coupled to the monitoring unit. The patient measuring system comprises monitoring unit, and the remote system may comprise the monitoring unit, or both may comprise the monitoring unit.

The external device can be configured to communicate with at least one of the adherent device or the monitoring unit. For example, the external device can be configured to communication directly with the adherent device, and the adherent device configured to communicate with the monitoring unit. Also, each of the external device and the adherent device can be configured to communicate directly with the monitoring unit.

In many embodiments, the system further comprises a notification device coupled to at least one of the patient measuring system or the remote monitoring system. The notification device is configured to provide notification when at least one value received from the plurality of sensors is outside an acceptable physiological range. At least one of the patient measuring system or the remote monitoring system may comprise the notification device. In some embodiments, the patient measuring system may comprises the notification device, and in additional embodiments the remote monitoring system may comprise the notification device, and in further embodiments both comprise the notification device.

In many embodiments, the monitoring unit comprises the notification device, and the notification device is located at the monitoring unit.

In many embodiments, the notification device comprises at least one of a visible patient indicator, an audible alarm, an emergency medical service notification, a call center alert, or direct medical provider notification. Each of these can emphasize the condition of the patient. For example, the notification device can be configured to provide notification to at least one of, the patient, a clinician, the remote monitoring system, a spouse, a family member, a caregiver or a medical provider and the notification device can be configured to transmit the notification from the notification device to another device, such as a therapeutic device. The notification device can be configured to notify two or more of the, patient, clinician, the remote monitoring system, a spouse, a family member, a caregiver or a medical provider, according to a preset hierarchy. The preset hierarchy may comprise patient notification first and medical provider second, or the preset hierarchy may comprise patient notification second and medical provider first.

In many embodiments, upon receipt of a notification, at least one of the medical provider, the remote monitoring system or a medical treatment device can trigger a high-rate sampling of physiological parameters for alert verification. The values received from the plurality of sensors are not within acceptable physiological ranges the notifying system communicates with the at least one of, the patient, a clinician, a spouse, a family member, a caregiver or a medical provider and from the notification device to another device to allow for therapeutic intervention to prevent decompensation.

In many embodiments, the system further comprises a processor system, and the processor system is configured to store a predetermined target value of the physiological parameters and a series of questions. The processor system can be configured to compare a sensor measured physiological parameter with the predetermined target value to determine a variance. The processor system may comprise a processor of the patient detecting system and a processor of the remote monitoring system, and the processor of the patient detecting system can be configured to accept and store a new predetermined target value and a new series of questions from the processor of the remote monitoring system.

In many embodiments, the patient detecting system includes at least one of a keyboard, a plurality of buttons or a microphone.

In another aspect, embodiments of the present invention provide a method of monitoring a patient to detect at least one physiological event. An adherent device is adhered to a patient to couple the device to the patient. The adherent device comprises a plurality of sensors for measuring patient data to detect the at least one physiological event of the patient. The patient data is transferred directly or indirectly from the plurality of sensors to a remote monitoring system with a wireless communication device coupled to the plurality of sensors. The remote monitoring system coupled to the wireless communication device, and the remote monitoring system comprises logic resources to determine the physiological event of the patient.

### BRIEF DESCRIPTION OF THE DRAWINGS

FIG. 1 is a block diagram illustrating one embodiment of a patient monitoring system of the present invention;

FIGS. 2A and 2B illustrate an exploded view and side view of embodiments of an adherent device with sensors configured to be coupled to the skin of a patient for monitoring purposes;

FIG. 3 illustrates one embodiment of an energy management device that is coupled to the plurality of sensors of FIG. 1;

FIG. 4 illustrates one embodiment of present invention illustrating logic resources configured to receive data from the sensors and/or the processed patient for monitoring purposes, analysis and/or prediction purposes;

FIG. 5 illustrates an embodiment of the patient monitoring system of the present invention with a memory management device;

FIG. 6 illustrates an embodiment of the patient monitoring system of the present invention with an external device coupled to the sensors;

FIG. 7 illustrates an embodiment of the patient monitoring system of the present invention with a notification device;

FIG. 8 is a block diagram illustrating an embodiment of the present invention with sensor leads that convey signals from the sensors to a monitoring unit at the detecting system, or through a wireless communication device to a remote monitoring system;

FIG. 9 is a block diagram illustrating an embodiment of the present invention with a control unit at the detecting system and/or the remote monitoring system;

FIG. 10 is a block diagram illustrating an embodiment of the present invention where a control unit encodes patient data and transmits it to a wireless network storage unit at the remote monitoring system;

FIG. 11 is a block diagram illustrating one embodiment of an internal structure of a main data collection station at the remote monitoring system of the present invention; and

FIG. 12 is a flow chart illustrating an embodiment of the present invention with method of operation steps performed by the system of the present invention in transmitting information to the main data collection station.

### DETAILED DESCRIPTION OF THE INVENTION

Embodiments of the present invention comprise a heart failure patient management system, which system may comprise an adherent patch device that is applied to the patient. The patch device continuously monitors physiological parameters, communicates wirelessly with a remote center, and provides alerts when necessary. A variety of communication configurations, notifications, and alerts can be used. The heart failure patient management system can monitor physiological parameters and may use an algorithm to determine heart failure status and predict impending decompensation. The system comprises an adherent patch configured with wireless communication capabilities. The patch device communicates with a remote center, in many embodiments via an intermediate device in the patient's home, such as a wireless gateway.

The system may include other external devices, such as a weight scale and blood pressure cuff, which communicate directly with the intermediate device or indirectly through the patch itself. In this way, communication with the remote center is centralized to a single device.

The system can monitor the patient's physiological parameters to determine heart failure status and predict impending decompensation. When notification is required, the system may direct notification to one or more of the following targets: the patient, clinician, remote call center (which may be either a server or a technician), spouse/family member, caregiver, and/or device-to-device (e.g. on/in body, modem, etc.).

In many embodiments, the system uses at least one of emphatic monitoring or emphatic validation, such that the remote monitoring system is aware of and sensitive to the thoughts and feelings of the patient, for example with a patient input device that allows the patient to enter at least one of his or her thoughts or feelings into the device. In response to a detection, or possible detection, of a physiological event, the remote monitoring system can contact, or solicit, the patient to see how her or she is doing and gather empathic, subjective data that may not be readily captured by the plurality of sensors, for example the patient's thoughts and feelings. The input device may comprise a hand held device in which the patient can type in his or her thoughts and feelings, for example a blackberry device. In some embodiments, the adherent patch may comprise a button switch or other sensor that the patient can tap, for example repeatedly to indicate that he or she is feeling very badly, for example in distress, and needs help. The emphatic monitoring from the remote site may also comprise a call center, in which personnel at the remote site and remote monitoring system can call the patient to ask how he or she is doing.

Notification may occur according to a preset hierarchy, such as in the following example:

1. Patient notification first (system connection verification, sensor accuracy verification, “how are you feeling?”, empathic validation).

2. Physician/nurse notification second, once false positives have been excluded.

One or more of a variety of notification types may be used. Notification may consist of a visible patient indicator (e.g. traffic light LED display), an audible alarm, emergency medical service notification (e.g. 911 call), a call center alert, or direct physician notification (e.g. page, fax, e-mail, web alert, etc.).

Upon initiation of an alert, the physician, remote center, and/or device may have the ability to trigger high-rate sampling of physiological parameters for alert verification. This high sampling rate of physiological parameters can further provide emphatic monitoring of the patient as such parameters can be indicative of how the patient is feeling. The physician, remote center, and/or device may also contact, or solicit, the patient to see how he or she is doing, for example by calling the patient and asking so as to confirm notification.

Many embodiments of the present invention provide heart failure patient monitoring. However, the system may be applicable to any human application in which wireless physiological monitoring is required.

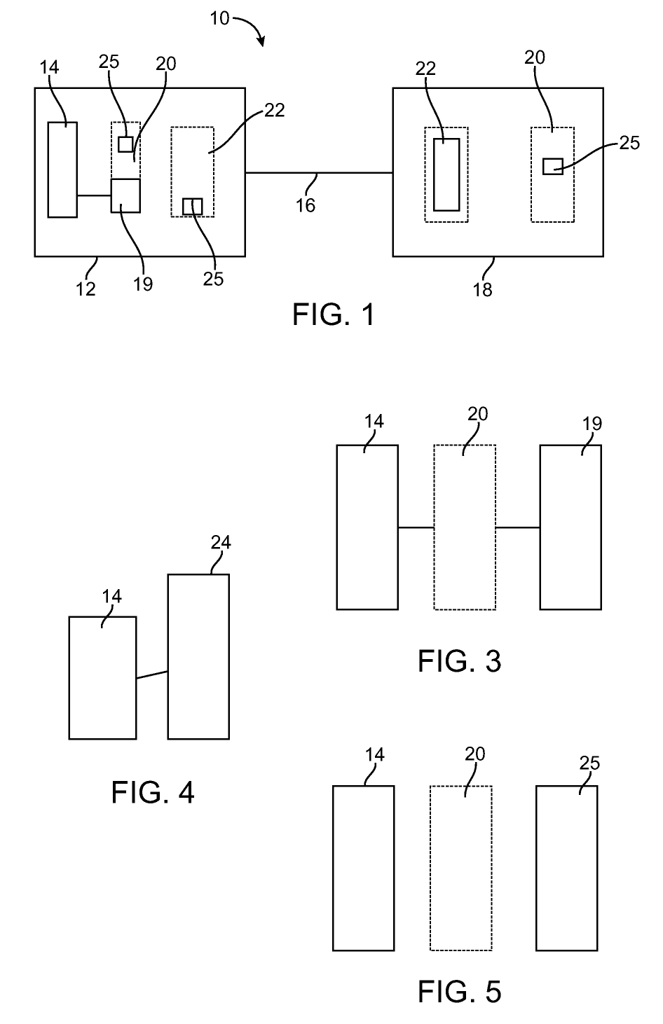

In one embodiment, illustrated in FIG. 1, the present invention is a patient management system, generally denoted as 10, that tracks the patient's physiological status, detects and predicts negative physiological events. In one embodiment, a plurality of sensors are used in combination to enhance detection and prediction capabilities as more fully explained below.

In one specific embodiment, the system 10 is used for decompensation prediction of a heart failure patient. For example system 10 may comprise a heart failure patient management system used for decompensation prediction of a heart failure patient. System 10 comprises a detecting system, for example a patient measuring system, denoted as 12, and a remote monitoring system 18. The detecting system comprises an adherent device configured to couple to the patient, for example configured to adhere to the patient's skin.

The adherent device comprises a plurality of sensors 14. The plurality of sensors can measure physiological parameters of the patient to monitor the patient and determine the status of the patient, for example to determine heart failure status. The physiological parameters can provide an indication of at least one physiological event, for example a cardiac decompensation or an impending cardiac decompensation. The plurality of sensors may be coupled to the patient, for example adhered to the patient's thorax. The adherent device may be housed in a tamper proof housing prior to placement on the patient.

The plurality of sensors 14 may comprise sensors to detect empathic patient input, such that the patient can provide input with respect to how he or she is feeling. The plurality of sensors may comprise a handheld device that allows the patient to enter at least one thoughts or feelings and transmit this data to the remote system. The plurality of sensors 14 may comprise buttons that the patient can press to indicate that he or she needs immediate assistance or is not feeling well.

The circuitry may comprise at least one of a processor, a processor system, logic circuitry or logic resources, can be configured in many ways to detect the at least one physiological event, such as heart failure. The circuitry may comprise discrete logic gates, such as and switch, programmable array logic, and microprocessor logic embodied in a tangible medium. For example, the remote monitoring system may comprise the logic circuitry, and the remote monitoring system may determine HF status when a rate of change of at least two sensor outputs comprises an abrupt change in the sensor outputs, such as an abrupt change as compared to a change in the sensor outputs over a longer period of time. The remote monitoring system may determine HF status by a tiered combination of at least a first and a second sensor output, with the first sensor output indicating a problem that is then verified by at least a second sensor output. The remote monitoring system may determine HF status in response a variance from a baseline value of sensor outputs. In some embodiments, the baseline values may be defined by a look up table. The HF status may be determined when a first sensor output is at a high value that is greater than a baseline value, and at least one of a second or a third sensor outputs is at a high value also sufficiently greater than a baseline value to indicate heart failure status. Heart failure status may be determined by time weighting the outputs of at least first, second and third sensors, and the time weighting indicates a recent event that is indicative of the heart failure status. When the patient measuring system comprises the logic circuitry, the patient measuring system may similarly detect the at least one physiological event.

The detecting system 12 also includes a wireless communication device 16, coupled to the plurality of sensors 14. The wireless communication device transfers patient data directly or indirectly from the plurality of sensors 14 to a remote monitoring system 18. The remote monitoring system 18 uses data from the sensors to determine heart failure status and predict impending decompensation of the patient. The detecting system 12 can continuously, or non-continuously, monitor the patient, alerts are provided as necessary and medical intervention is provided when required. The wireless communication device 16 may comprise at least one of a gateway or a wireless local area network for receiving data from the plurality of sensors.

The plurality of sensors 14 may comprise at least one ID sensor. The at least one ID sensor may be coupled to the adherent device, addressable, and unique to each adherent device. The adherent device may comprise the ID sensor of the plurality of sensors 14.

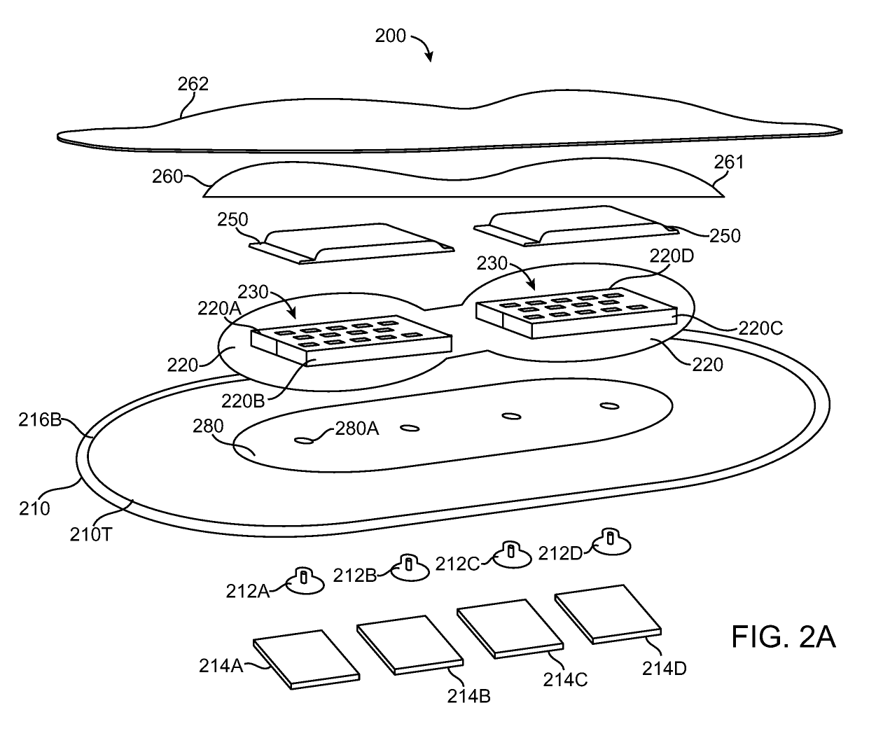

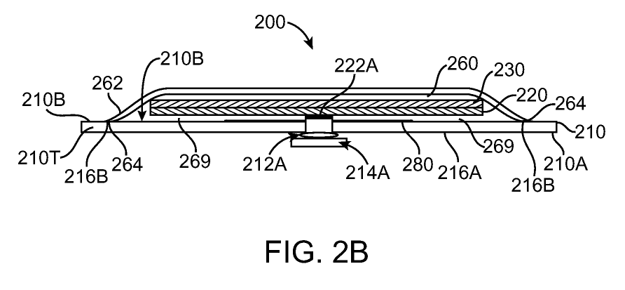

FIGS. 2A and 2B show embodiments of the plurality of sensors 14 with supported with an adherent device 200 configured to adhere to the skin. Adherent device 200 is described in U.S. App. No. 60/972,537, the full disclosure of which has been previously incorporated herein by reference. As illustrated in an exploded view of the adherent device, a cover 262, batteries 250, electronics 230, including but not limited to flex circuits and the like, an adherent tape 210T, the plurality of sensors may comprise electrodes and sensor circuitry, and hydrogels which interface the plurality of sensors 14 with the skin, are provided.

Adherent device 200 comprises a support, for example adherent patch 210, configured to adhere the device to the patient. Adherent patch 210 comprises a first side, or a lower side 210A, that is oriented toward the skin of the patient when placed on the patient and a second side, or upper side 210B, opposite of the first side. In many embodiments, adherent patch 210 comprises a tape 210T which is a material, preferably breathable, with an adhesive 216A. Patient side 210A comprises adhesive 216A to adhere the patch 210 and adherent device 200 to patient P. Electrodes 212A, 212B, 212C and 212D are affixed to adherent patch 210. In many embodiments, at least four electrodes are attached to the patch, for example six electrodes. In some embodiments the patch comprises two electrodes, for example two electrodes to measure the electrocardiogram (ECG) of the patient. Gel 214A, gel 214B, gel 214C and gel 214D can each be positioned over electrodes 212A, 212B, 212C and 212D, respectively, to provide electrical conductivity between the electrodes and the skin of the patient. In many embodiments, the electrodes can be affixed to the patch 210, for example with known methods and structures such as rivets, adhesive, stitches, etc. In many embodiments, patch 210 comprises a breathable material to permit air and/or vapor to flow to and from the surface of the skin. In some embodiments, a printed circuit board (PCB), for example flex PCB 220, may be connected to upper side 210B of patch 210 with connectors. In some embodiments, additional PCB's, for example rigid PCB's 220A, 220B, 220C and 220D, can be connected to flex PCB 220. Electronic components 230 can be connected to flex PCB 220 and/or mounted thereon. In some embodiments, electronic components 230 can be mounted on the additional PCB's.

Electronic circuitry and components 230 comprise circuitry and components to take physiologic measurements, transmit data to remote center and receive commands from remote center. In many embodiments, electronics components 230 may comprise known low power circuitry, for example complementary metal oxide semiconductor (CMOS) circuitry components. Electronics components 230 comprise an activity sensor and activity circuitry, impedance circuitry and electrocardiogram circuitry, for example ECG circuitry. In some embodiments, electronics circuitry may comprise a microphone and microphone circuitry to detect an audio signal from within the patient, and the audio signal may comprise a heart sound and/or a respiratory sound, for example an S3 heart sound and a respiratory sound with rales and/or crackles. Electronics circuitry and components 230 may comprise a temperature sensor, for example a thermistor, and temperature sensor circuitry to measure a temperature of the patient, for example a temperature of a skin of the patient.

A cover 262 can extend over the batteries, electronic components and flex printed circuit board. In many embodiments, an electronics housing 260 may be disposed under cover 262 to protect the electronic components, and in some embodiments electronics housing 260 may comprise an encapsulant over the electronic components and PCB. In some embodiments, cover 262 can be adhered to adhesive patch with an adhesive. In many embodiments, electronics housing 260 may comprise a water proof material, for example a sealant adhesive such as epoxy or silicone coated over the electronics components and/or PCB. In some embodiments, electronics housing 260 may comprise metal and/or plastic. Metal or plastic may be potted with a material such as epoxy or silicone.

Cover 262 may comprise many known biocompatible cover, casing and/or housing materials, such as elastomers, for example silicone. The elastomer may be fenestrated to improve breathability. In some embodiments, cover 262 may comprise many known breathable materials, for example polyester, polyamide, and/or elastane (Spandex). The breathable fabric may be coated to make it water resistant, waterproof, and/or to aid in wicking moisture away from the patch.

Adherent device 200 comprises several layers. Gel 214A, or gel layer, is positioned on electrode 212A to provide electrical conductivity between the electrode and the skin. Electrode 212A may comprise an electrode layer. Adhesive patch 210 may comprise a layer of breathable tape 210T, for example a known breathable tape, such as tricot-knit polyester fabric. In many embodiments, a gap 269 extends from adhesive patch 210 to the electronics circuitry and components 230, such that breathable tape 210T can breath to provide patient comfort. An adhesive 216A, for example a layer of acrylate pressure sensitive adhesive, can be disposed on underside 210A of patch 210. A gel cover 280, or gel cover layer, for example a polyurethane non-woven tape, can be positioned over patch 210 comprising the breathable tape. A PCB layer, for example flex PCB 220, or flex PCB layer, can be positioned over gel cover 280 with electronic components 230 connected and/or mounted to flex PCB 220, for example mounted on flex PCB so as to comprise an electronics layer disposed on the flex PCB. In many embodiments, the adherent device may comprise a segmented inner component, for example the PCB, for limited flexibility. In many embodiments, the electronics layer may be encapsulated in electronics housing 260 which may comprise a waterproof material, for example silicone or epoxy. In many embodiments, the electrodes are connected to the PCB with a flex connection, for example trace 222A of flex PCB 220, so as to provide strain relief between the electrodes 212A, 212B, 212C and 212D and the PCB. Gel cover 280 can inhibit flow of gel 214A and liquid. In many embodiments, gel cover 280 can inhibit gel 214A from seeping through breathable tape 210T to maintain gel integrity over time. Gel cover 280 can also keep external moisture from penetrating into gel 214A. Gel cover 280 may comprise at least one aperture 280A sized to receive one of the electrodes. In many embodiments, cover 262 can encase the flex PCB and/or electronics and can be adhered to at least one of the electronics, the flex PCB or the adherent patch, so as to protect the device. In some embodiments, cover 262 attaches to adhesive patch 210 with adhesive 216B or adhesive 264. Cover 262 can comprise many known biocompatible cover, housing and/or casing materials, for example silicone. In many embodiments, cover 262 comprises an outer polymer cover to provide smooth contour without limiting flexibility. In some embodiments, cover 262 may comprise a breathable fabric. Cover 262 may comprise many known breathable fabrics, for example breathable fabrics as described above. In some embodiments, the breathable fabric may comprise polyester, polyamide, and/or elastane (Spandex™) to allow the breathable fabric to stretch with body movement. In some embodiments, the breathable tape may contain and elute a pharmaceutical agent, such as an antibiotic, anti-inflammatory or antifungal agent, when the adherent device is placed on the patient.

In one embodiment, the wireless communication device 16 is configured to receive instructional data from the remote monitoring system.

Referring to FIG. 3, an energy management device 19 can be coupled to the plurality of sensors. In one embodiment, the energy management device 19 is part of the detecting system. In various embodiments, the energy management device 19 performs one or more of modulate drive levels per sensed signal of a sensor 14, modulate a clock speed to optimize energy, watch cell voltage drop—unload cell, coulomb-meter or other battery monitor, sensor dropoff at an end of life of a battery coupled to a sensor, battery end of life dropoff to transfer data, elective replacement indicator, call center notification, sensing windows by the sensors 14 based on a monitored physiological parameter and sensing rate control.

In one embodiment, energy management is achieved by using time as a variable. This can be achieved by intermittent sampling. Variable time courses can be used for measuring signals from the beginning and the duty cycle rates can be adjusted, for example adjusted at the remote monitoring system 18.

In one embodiment, the energy management device 19 is configured to manage energy by at least one of, a thermo-electric unit, kinetics, fuel cell, through solar power, a zinc air interface, Faraday generator, internal combustion, nuclear power, a micro-battery and with a rechargeable device.

In one embodiment, the adherent device includes a patch set configured to be coupled to the patient. Patches in the patch set, as well as replacement patches can be linked together and coupled to hardware at the detecting system 12 or at the remote monitoring system 18. Patches of the patch set can also be linked at software at a back end at the remote monitoring system 18. Registration with the remote monitoring system 18 can occur each time a new patch is put on the patient.

When an adherent device is provided to a patient, a medical provider registers that adherent device, associated with that patient, with the remote monitoring system 18. Registration can take place a variety of different ways, including but not limited to, via a web site, and the like. Upon registration, patient data is uploaded to the adherent device. An association of the adherent patch with the patient occurs by at least one of, caller ID, an RFID tag on the patient, a body tattoo, fingerprint ID and GPS.

In one embodiment, a modem is assigned to the patient and links to the adherent device. The modem can be configured to determine which patch is sending information to the modem. The modem communicates only with the patch set of the patient, and the modem only communicates with those patches with which it is associated. The modem can be at the detecting system 12 or at the remote monitoring system 18.

In one embodiment, the ID sensor 14 has a removable memory component with a unique patient ID that is reused as patches of the patch set are replaced. In one embodiment, the ID sensor 14 produces a first output that has protected patient data with restricted communication, and a second output that has general device and patient information for general communication. Access to the protected patient data can require an additional security verification. At least a portion of the protected patient data can be encrypted. A variety of additional security verifications including but not limited to, a skin tattoo with an adherent device reader, a modem identification, an encrypted communication, an encrypted data storage on the adherent device, a biometric ID, an x-ray ID tag and the like.

The system 10 is configured to automatically detect events. The system 10 automatically detects events by at least one of, high noise states, physiological quietness, sensor continuity and compliance. In response to a detected physiological event, patient states are identified when data collection is inappropriate. In response to a detected physiological event, patient states are identified when data collection is desirable. Patient states include, physiological quietness, rest, relaxation, agitation, movement, lack of movement and a patient's higher level of patient activity.

The system can use an intelligent combination of sensors to enhance detection and prediction capabilities, as more fully discloses in U.S. patent application Ser. No. 60/972,537, identified as Attorney Docket No. 026843-000200US, filed Sep. 14, 2007, the full disclosure of which has been previously incorporated herein by reference, and as more fully explained below. The intelligent combination of sensors may comprise sensor to measure at least two of an electrocardiogram signal, a hydration signal, an accelerometer signal or a respiration signal of the patient.

In one embodiment, the detecting system 12 communicates with the remote monitoring system 18 periodically or in response to a trigger event. The trigger event can include but is not limited to at least one of, time of day, if a memory is full, if an action is patient initiated, if an action is initiated from the remote monitoring system, a diagnostic event of the monitoring system, an alarm trigger, a mechanical trigger, and the like.

The adherent device be activated by a variety of different means including but not limited to, a physiological trigger, automatic impedance, a tab pull, battery insertion, a hall or reed switch, a breakable glass capsule, a dome switch, by light activation, pressure activation, body temperature activation, a connection between electronics associated with the sensors and the adherent device, exposure to air, by a capacitive skin sensor and the like.

The detecting system 12 can continuously, or non-continuously, monitor the patient, alerts are provided as necessary and medical intervention is provided when required. In one embodiment, the wireless communication device 16 is a wireless local area network for receiving data from the plurality of sensors.

At least one at least one processor 20 is coupled to the plurality of sensors 14 and can also be a part of the wireless communication device 16. The at least one at least one processor 20 comprises at least one tangible medium and may comprise a processor system. The at least one at least one processor 20 receives data from the plurality of sensors 14 and creates processed patient data.

In many embodiments, the at least one processor 20 comprises at least one of a processor of detecting system 12 comprising a tangible medium, a processor of remote monitoring system 18 comprising a tangible medium, a processor of wireless communication device 16 comprising a tangible medium or a processor of monitoring unit 22 comprising a tangible medium. In one embodiment, the at least one at least one processor 20 is located at the remote monitoring system. In another embodiment, the at least one at least one processor 20 is located at the detecting system 12.

The at least one processor 20 can be integral with a monitoring unit 22 that is part of the detecting system 12 or part of the remote monitoring system, or both. The monitoring unit can be located at the remote monitoring system 18.

The at least one processor 20 has program instructions for evaluating values received from the sensors 14 with respect to acceptable physiological ranges for each value received by the at least one processor 20 and determine variances. The at least one processor 20 can receive and store a sensed measured parameter from the sensors 14, compare the sensed measured value with a predetermined target value, determine a variance, accept and store a new predetermined target value and also store a series of questions from the remote monitoring system 18.

As shown in FIG. 4, logic resources 24 are provided that take the data from the sensors 14, and/or the processed patient data from the at least one processor 20, to predict an impending decompensation. The logic resources 24 can be at the remote monitoring system 18 or at the detecting system 12, such as in the monitoring unit 22.

In one embodiment, illustrated in FIG. 5, a memory management device 25 is provided. In various embodiments, the memory management device 25 performs one or more of data compression, prioritizing of sensing by a sensor 14, monitoring all or some of sensor data by all or a portion of sensors 14, sensing by the sensors 14 in real time, noise blanking to provide that sensor data is not stored if a selected noise level is determined, low-power of battery caching and decimation of old sensor data.

The sensors 14 can provide a variety of different functions, including but not limited to, initiation, programming, measuring, storing, analyzing, communicating, predicting, and displaying of a physiological event of the patient. Each of sensors 14 is preferably sealed, such as housed in a hermetically sealed package. In one embodiment, at least a portion of the sealed packages include a power source, a memory, logic resources and a wireless communication device. In one embodiment, the sensors 14 can include, flex circuits, thin film resistors, organic transistors and the like. The sensors 14 can include ceramics to enclose the electronics. Additionally, the sensors 14 can include drug eluting coatings, including but not limited to, an antibiotic, anti-inflammatory agent and the like.

A wide variety of different sensors 14 can be utilized, including but not limited to, bioimpedance, heart rate, heart rhythm, HRV, HRT, heart sounds, respiration rate, respiration rate variability, respiratory sounds, SpO2, blood pressure, activity, posture, wake/sleep, orthopnea, temperature, heat flux and an accelerometer. A variety activity sensors can be utilized, including but not limited to a, ball switch, accelerometer, minute ventilation, HR, bioimpedance noise, skin temperature/heat flux, BP, muscle noise, posture and the like.

The outputs of the sensors 14 can have multiple features to enhance physiological sensing performance. These multiple features have multiple sensing vectors that can include redundant vectors. The sensors can include current delivery electrodes and sensing electrodes. Size and shape of current delivery electrodes, and the sensing electrodes, can be optimized to maximize sensing performance. The system 10 can be configured to determine an optimal sensing configuration and electronically reposition at least a portion of a sensing vector of a sensing electrode. The multiple features enhance the ability of system 10 to determine an optimal sensing configuration and electronically reposition sensing vectors. In one embodiment, the sensors 14 can be partially masked to minimize contamination of parameters sensed by the sensors 14.

The size and shape of current delivery electrodes, for bioimpedance, and sensing electrodes can be optimized to maximize sensing performance. Additionally, the outputs of the sensors 14 can be used to calculate and monitor blended indices. Examples of the blended indices include but are not limited to, heart rate (HR) or respiratory rate (RR) response to activity, HR/RR response to posture change, HR+RR, HR/RR+bioimpedance, and/or minute ventilation/accelerometer and the like.

The sensors 14 can be cycled in order to manage energy, and different sensors 14 can sample at different times. By way of illustration, and without limitation, instead of each sensor 14 being sampled at a physiologically relevant interval, e.g. every 30 seconds, one sensor 14 can be sampled at each interval, and sampling cycles between available sensors.

By way of illustration, and without limitation, the sensors 14 can sample 5 seconds for every minute for ECG, once a second for an accelerometer sensor, and 10 seconds for every 5 minutes for impedance.

In one embodiment, a first of sensors 14 comprises a core sensor that continuously monitors and detects, and a second of sensors 14 verifies a physiological status in response to the core sensor 14 raising a flag. Additionally, at least some of sensors 14 can be used for short term tracking, and other sensors of sensor 14 used for long term tracking.

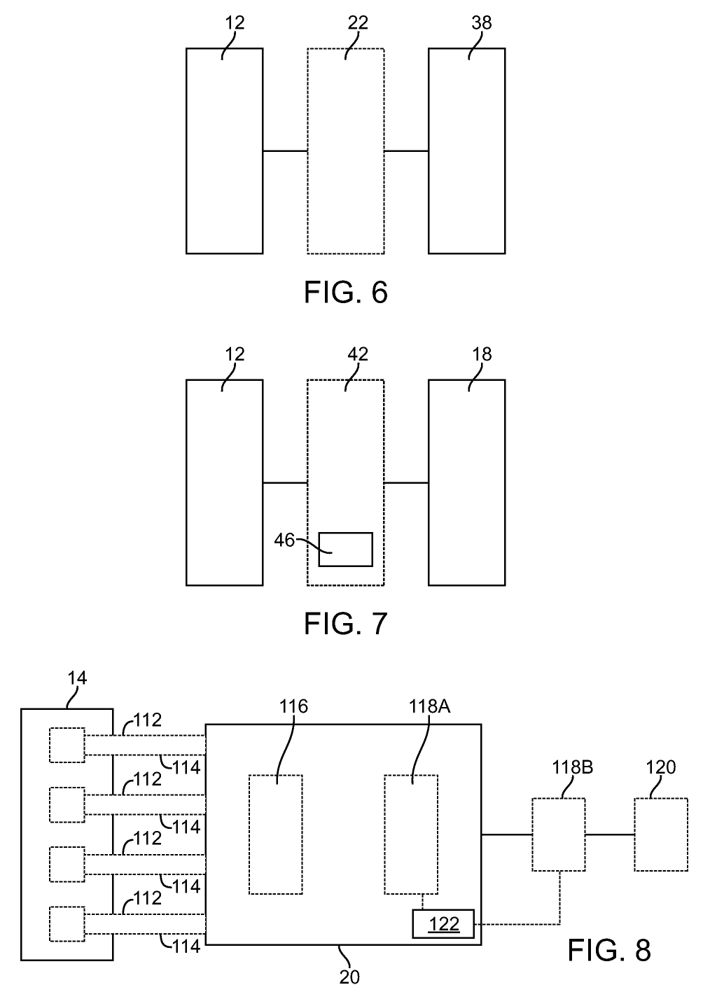

Referring to FIG. 6, in one embodiment, an external device 38, which may comprise a medical treatment device, is coupled to the sensors 14. The external device 38 can be coupled to a monitoring unit 22 that is part of the detecting system 12, or in direct communication with the sensors 14. A variety of different external devices 38 can be used to monitor and/or treat the patient, the external devices 38 including but not limited to, a weight scale, blood pressure cuff, cardiac rhythm management device, a medical treatment device, medicament dispenser and the like). Suitable cardiac rhythm management devices include but are not limited to, Boston Scientific's Latitude system, Medtronic's C are Link system, St. Jude Medical's HouseCall system and the like. Such communication may occur directly, or via an external translator unit.

The external device 38 can be coupled to an auxiliary input of the monitoring unit 22 at the detecting system 12 or to the monitoring system 22 at the remote monitoring system 18. Additionally, an automated reader can be coupled to an auxiliary input in order to allow a single monitoring unit 22 to be used by multiple patients. As previously mentioned above, the monitoring unit 22 can be at the remote monitoring system 18 and each patient can have a patient identifier (ID) including a distinct patient identifier. In addition, the ID identifier can also contain patient specific configuration parameters. The automated reader can scan the patient identifier ID and transmit the patient ID number with a patient data packet such that the main data collection station can identify the patient.

It will be appreciated that other medical treatment devices can also be used. The sensors 14 can communicate wirelessly with the external devices 38 in a variety of ways including but not limited to, a public or proprietary communication standard and the like. The detecting system 12 comprising sensors 14 can be configured to serve as a communication hub for multiple medical devices, coordinating sensor data and therapy delivery while transmitting and receiving data from the remote monitoring system 18.

In one embodiment, the detecting system 12 comprising sensors 14 is configured to coordinate data sharing between the external systems 38 allowing for sensor integration across devices. The coordination of the sensors 14 provides for new pacing, sensing, defibrillation vectors and the like.

In one embodiment, the at least one processor 20 is included in the monitoring unit 22 and the external device 38 is in direct communication with the monitoring unit 22.

In another embodiment, illustrated in FIG. 7, a notification device 42 is coupled to the detecting system 12 and the remote monitoring system 18. The notification device 42 is configured to provide notification when values received from the sensors 14 are not within acceptable physiological ranges. The notification device 42 can be at the remote monitoring system 18 or at the monitoring unit 22 that is part of the detecting system 12. A variety of notification devices 42 can be utilized, including but not limited to, a visible patient indicator, an audible alarm, an emergency medical service notification, a call center alert, direct medical provider notification and the like. The notification device 42 provides notification to a variety of different entities, including but not limited to, the patient, a caregiver, the remote monitoring system, a spouse, a family member, a medical provider, from one device to another device such as the external device 38, and the like.

Notification can be according to a preset hierarchy. By way of illustration, and without limitation, the preset hierarchy can be, patient notification first and medical provider second, patient notification second and medical provider first, and the like. Upon receipt of a notification, a medical provider, the remote monitoring system 18, or a medical treatment device can trigger a high-rate sampling of physiological parameters for alert verification.

The system 10 can also include an alarm 46, that can be coupled to the notification device 42, for generating a human perceptible signal when values received from the sensors 14 are not within acceptable physiological ranges. The alarm 46 can trigger an event to render medical assistance to the patient, provide notification as set forth above, continue to monitor, wait and see, and the like.

When the values received from the sensors 14 are not within acceptable physiological ranges the notification is with the at least one of, the patient, a spouse, a family member, a caregiver, a medical provider and from one device to another device, to allow for therapeutic intervention to prevent decompensation, and the like.

In another embodiment, the sensors 14 can switch between different modes, wherein the modes are selected from at least one of, a stand alone mode with communication directly with the remote monitoring system 18, communication with an implanted device, communication with a single implanted device, coordination between different devices (external systems) coupled to the plurality of sensors and different device communication protocols.

By way of illustration, and without limitation, the patient can be a congestive heart failure patient. Heart failure status is determined by a weighted combination change in sensor outputs and be determined by a number of different means, including but not limited to, (i) when a rate of change of at least two sensor outputs is an abrupt change in the sensor outputs as compared to a change in the sensor outputs over a longer period of time, (ii) by a tiered combination of at least a first and a second sensor output, with the first sensor output indicating a problem that is then verified by at least a second sensor output, (iii) by a variance from a baseline value of sensor outputs, and the like. The baseline values can be defined in a look up table.

In another embodiment, heart failure status is determined using three or more sensors by at least one of, (i) when the first sensor output is at a value that is sufficiently different from a baseline value, and at least one of the second and third sensor outputs is at a value also sufficiently different from a baseline value to indicate heart failure status, (ii) by time weighting the outputs of the first, second and third sensors, and the time weighting indicates a recent event that is indicative of the heart failure status and the like.

In one embodiment, the wireless communication device 16 can include a, modem, a controller to control data supplied by the sensors 14, serial interface, LAN or equivalent network connection and a wireless transmitter. Additionally, the wireless communication device 16 can include a receiver and a transmitter for receiving data indicating the values of the physiological event detected by the plurality of sensors, and for communicating the data to the remote monitoring system 18. Further, the wireless communication device 16 can have data storage for recording the data received from the sensors 14 and an access device for enabling access to information recording in the data storage from the remote monitoring system 18.

In various embodiments, the remote monitoring system 18 can include a receiver, a transmitter and a display for displaying data representative of values of the one physiological event detected by the sensors 14. The remote monitoring system can also include a, data storage mechanism that has acceptable ranges for physiological values stored therein, a comparator for comparing the data received from the monitoring system 12 with the acceptable ranges stored in the data storage device and a portable computer. The remote monitoring system 18 can be a portable unit with a display screen and a data entry device for communicating with the wireless communication device 16.

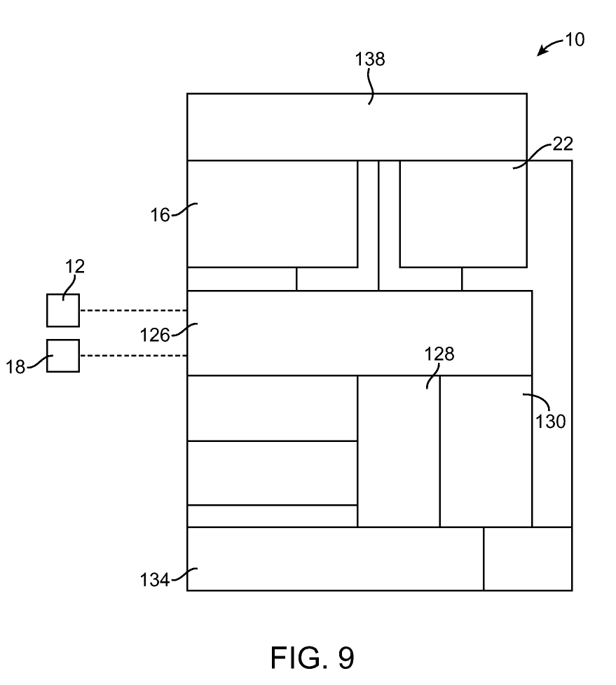

Referring now to FIG. 8, for each of sensors 14, a sensor lead 112 and 114 conveys signals from the sensor 14 to the monitoring unit 22 at the detecting system 12, or through the wireless communication device 16 to the remote monitoring system 18, or both. In one embodiment, each signal from a sensor 14 is first passed through a low-pass filter 116, at the detecting system 12 or at the remote monitoring system 18, to smooth the signal and reduce noise. The signal is then transmitted to an analog-to-digital converter 118A, which transforms the signals into a stream of digital data values that can be stored in a digital memory 118B. From the digital memory 118B, data values are transmitted to a data bus 120, along which they are transmitted to other components of the circuitry to be processed and archived. From the data bus 120, the digital data can be stored in a non-volatile data archive memory. The digital data can be transferred via the data bus 120 to the at least one processor 20, which processes the data based in part on algorithms and other data stored in a non-volatile program memory.

The detecting system 12 can also include a power management module 122 configured to power down certain components of the system, including but not limited to, the analog-to-digital converters 118A, digital memories 118B and the non-volatile data archive memory and the like, between times when these components are in use. This helps to conserve battery power and thereby extend the useful life. Other circuitry and signaling modes may be devised by one skilled in the art.

As can be seen in FIG. 9, a control unit 126 is included at the detecting system 12, the remote monitoring system 18 or at both locations.

In one embodiment, the control unit 126 can be a known 486 microprocessor, available from Intel, Inc. of Santa Clara, Calif. The control unit 126 can be coupled to the sensors 14 directly at the detecting system 12, indirectly at the detecting system 12 or indirectly at the remote monitoring system 18. Additionally the control unit 126 can be coupled to a blood pressure monitor, a cardiac rhythm management device, a scale or a device that dispenses medication that can indicate the medication has been dispensed.

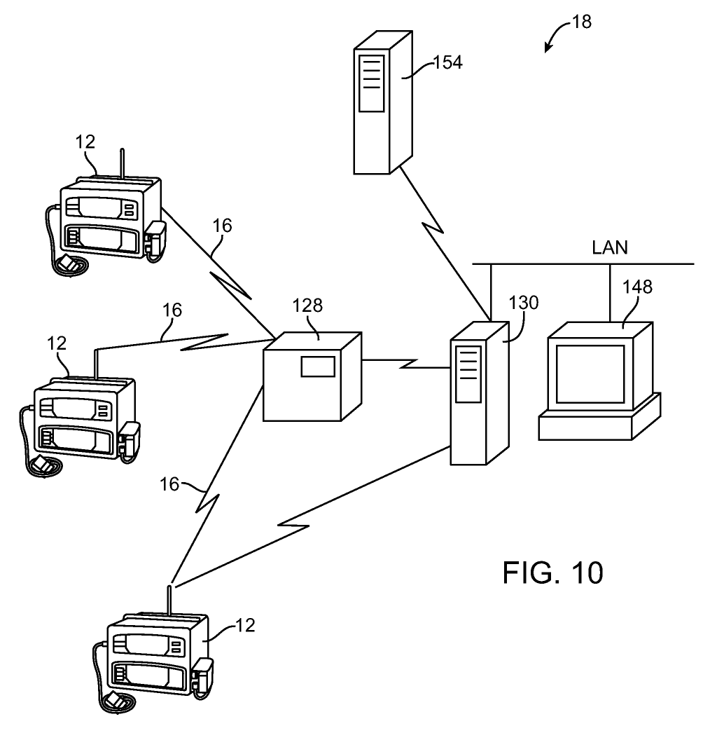

The control unit 126 can be powered by AC inputs which are coupled to internal AC/DC converters 134 that generate multiple DC voltage levels. After the control unit 126 has collected the patient data from the sensors 14, the control unit 126 encodes the recorded patient data and transmits the patient data through the wireless communication device 16 to transmit the encoded patient data to a wireless network storage unit 128 at the remote monitoring system 18 as shown in FIG. 10. In another embodiment, wireless communication device 16 transmits the patient data from the sensors 14 to the control unit 126 when it is at the remote monitoring system 18.

Each time the control unit 126 plans to transmit patient data to a main data collection station 130, located at the remote monitoring system 18, the control unit 126 attempts to establish a communication link. The communication link can be wireless, wired, or a combination of wireless and wired for redundancy, e.g., the wired link checks to see if a wireless communication can be established. If the wireless communication link 16 is available, the control unit 126 transmits the encoded patient data through the wireless communication device 16. However, if the wireless communication device 16 is not available for any reason, the control unit 126 waits and tries again until a link is established.

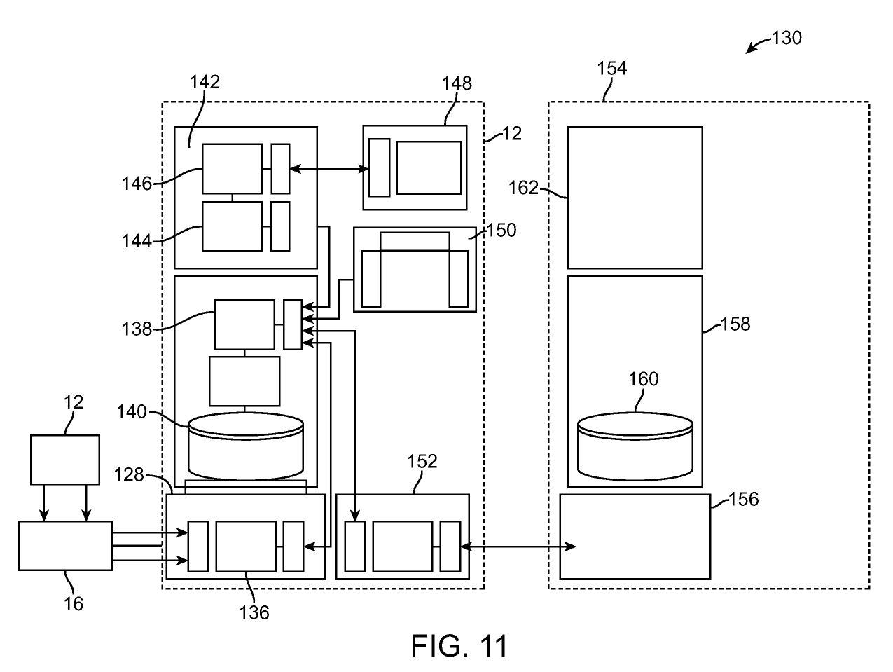

Referring now to FIG. 10 and FIG. 11, one embodiment of an internal structure of a main data collection station 130, at the remote monitoring system 18, is illustrated. The patient data can be transmitted to the remote monitoring system 18 by either the wireless communication device 16 or conventional modem to the wireless network storage unit 128. After receiving the patient data, the wireless network storage unit 128 can be accessed by the main data collection station 130. The main data collection station 130 allows the remote monitoring system 18 to monitor the patient data of numerous patients from a centralized location without requiring the patient or a medical provider to physically interact with each other.

The main data collection station 130 can include a communications server 136 that communicates with the wireless network storage unit 128. The wireless network storage unit 128 can be a centralized computer server that includes a unique, password protected mailbox assigned to and accessible by the main data collection station 130. The main data collection station 130 communicates with the wireless network storage unit 128 and downloads the patient data stored in a mailbox assigned to the main data collection station 130.

Once the communications server 136 has formed a link with the wireless network storage unit 128, and has downloaded the patient data, the patient data can be transferred to a database server 138. The database server 138 includes a patient database 140 that records and stores the patient data of the patients based upon identification included in the data packets sent by each of the monitoring units 22. For example, each data packet can include an identifier.

Each data packet transferred from the remote monitoring system 18 to the main data collection station 130 does not have to include any patient identifiable information. Instead, the data packet can include the serial number assigned to the specific detecting system 12. The serial number associated with the detecting system 12 can then be correlated to a specific patient by using information stored on the patient database 138. In this manner, the data packets transferred through the wireless network storage unit 128 do not include any patient-specific identification. Therefore, if the data packets are intercepted or improperly routed, patient confidentiality can not be breached.

The database server 138 can be accessible by an application server 142. The application server 142 can include a data adapter 144 that formats the patient data information into a form that can be viewed over a conventional web-based connection. The transformed data from the data adapter 144 can be accessible by propriety application software through a web server—146 such that the data can be viewed by a workstation 148. The workstation 148 can be a conventional personal computer that can access the patient data using proprietary software applications through, for example, HTTP protocol, and the like.

The main data collection station further can include an escalation server 150 that communicates with the database server 138. The escalation server 150 monitors the patient data packets that are received by the database server 138 from the monitoring unit 22. Specifically, the escalation server 150 can periodically poll the database server 138 for unacknowledged patient data packets. The patient data packets are sent to the remote monitoring system 18 where the processing of patient data occurs. The remote monitoring system 18 communicates with a medical provider if the event that an alert is required. If data packets are not acknowledged by the remote monitoring system 18. The escalation server 150 can be programmed to automatically deliver alerts to a specific medical provider if an alarm message has not been acknowledged within a selected time period after receipt of the data packet.

The escalation server 150 can be configured to generate the notification message to different people by different modes of communication after different delay periods and during different time periods.

The main data collection station 130 can include a batch server 152 connected to the database server 138. The batch server 152 allows an administration server 154 to have access to the patient data stored in the patient database 140. The administration server allows for centralized management of patient information and patient classifications.

The administration server 154 can include a batch server 156 that communicates with the batch server 152 and provides the downloaded data to a data warehouse server 158. The data warehouse server 158 can include a large database 160 that records and stores the patient data.

The administration server 154 can further include an application server 162 and a maintenance workstation 164 that allow personnel from an administrator to access and monitor the data stored in the database 160.

The data packet utilized in the transmission of the patient data can be a variable length ASCII character packet, or any generic data formats, in which the various patient data measurements are placed in a specific sequence with the specific readings separated by commas. The control unit 126 can convert the readings from each sensor 14 into a standardized sequence that forms part of the patient data packet. In this manner, the control unit 126 can be programmed to convert the patient data readings from the sensors 14 into a standardized data packet that can be interpreted and displayed by the main data collection station 130 at the remote monitoring system 18.

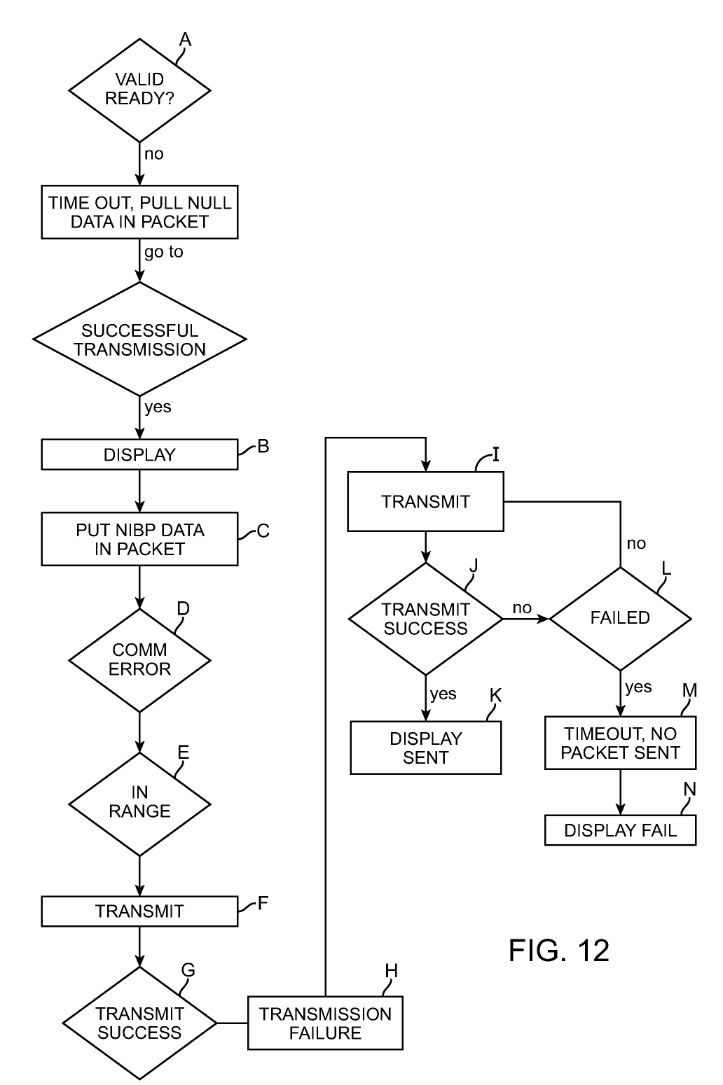

Referring now to the flow chart and method of operation shown in FIG. 12, if an external device 38 fails to generate a valid reading, as illustrated in step A, the control unit 126 fills the portion of the patient data packet associated with the external device 38 with a null indicator. The null indicator can be the lack of any characters between commas in the patient data packet. The lack of characters in the patient data packet can indicate that the patient was not available for the patient data recording. The null indicator in the patient data packet can be interpreted by the main data collection station 130 at the remote monitoring system 18 as a failed attempt to record the patient data due to the unavailability of the patient, a malfunction in one or more of the sensors 14, or a malfunction in one of the external devices 38. The null indicator received by the main data collection station 130 can indicate that the transmission from the detecting system 12 to the remote monitoring system 18 was successful. In one embodiment, the integrity of the data packet received by the main data collection station 130 can be determined using a cyclic redundancy code, CRC-16, check sum algorithm. The check sum algorithm can be applied to the data when the message can be sent and then again to the received message.

After the patient data measurements are complete, the control unit 126 displays the sensor data, including but not limited to blood pressure cuff data and the like, as illustrated by step B. In addition to displaying this data, the patient data can be placed in the patient data packet, as illustrated in step C.

As previously described, the system 10 can take additional measurements utilizing one or more auxiliary or external devices 38 such as those mentioned previously. Since the patient data packet has a variable length, the auxiliary device patient information can be added to the patient data packet being compiled by the remote monitoring unit 22 during patient data acquisition period being described. Data from the external devices 38 is transmitted by the wireless communication device 16 to the remote monitoring system 18 and can be included in the patient data packet.

If the remote monitoring system 18 can be set in either the auto mode or the wireless only mode, the remote monitoring unit 22 can first determine if there can be an internal communication error, as illustrated in step D.

A no communication error can be noted as illustrated in step E. If a communication error is noted the control unit 126 can proceed to wireless communication device 16 or to a conventional modem transmission sequence, as will be described below. However, if the communication device is working the control unit 126 can transmit the patient data information over the wireless network 16, as illustrated in step F. After the communication device has transmitted the data packet, the control unit 126 determines whether the transmission was successful, as illustrated in step G. If the transmission has been unsuccessful only once, the control unit 126 retries the transmission. However, if the communication device has failed twice, as illustrated in step H, the control unit 126 proceeds to the conventional modem process if the remote monitoring unit 22 was configured in an auto mode.

When the control unit 126 is at the detecting system 12, and the control unit 126 transmits the patient data over the wireless communication device 16, as illustrated in step I, if the transmission has been successful, the display of the remote monitoring unit 22 can display a successful message, as illustrated in step J. However, if the control unit 126 determines in step K that the communication of patient data has failed, the control unit 126 repeats the transmission until the control unit 126 either successfully completes the transmission or determines that the transmission has failed a selected number of times, as illustrated in step L. The control unit 126 can time out the and a failure message can be displayed, as illustrated in steps M and N. Once the transmission sequence has either failed or successfully transmitted the data to the main data collection station, the control unit 126 returns to a start program step, for example step A.

The processor system, as described above, can be configured to perform the method shown in FIG. 12, including many of the steps described above. It should be appreciated that the specific steps illustrated in FIG. 12 provide a particular method, according to one embodiment of the present invention. Other sequences of steps may also be performed according to alternative embodiments. For example, alternative embodiments of the present invention may perform the steps outlined above in a different order. Moreover, the individual steps illustrated in FIG. 12 may include multiple sub-steps that may be performed in various sequences as appropriate to the individual step. Furthermore, additional steps may be added or removed depending on the particular applications. One of ordinary skill in the art would recognize many variations, modifications, and alternatives.

As discussed previously, the patient data packets are first sent and stored in the wireless network storage unit 128. From there, the patient data packets are downloaded into the main data collection station 130. The main data collection station 130 decodes the encoded patient data packets and records the patient data in the patient database 140. The patient database 140 can be divided into individual storage locations for each patient such that the main data collection station 130 can store and compile patient data information from a plurality of individual patients.

A report on the patient's status can be accessed by a medical provider through a medical provider workstation that is coupled to the remote monitoring system 18. Unauthorized access to the patient database can be prevented by individual medical provider usernames and passwords to provide additional security for the patient's recorded patient data.

The main data collection station 130 and the series of work stations 148 allow the remote monitoring system 18 to monitor the daily patient data measurements taken by a plurality of patients reporting patient data to the single main data collection station 130. The main data collection station 130 can be configured to display multiple patients on the display of the workstations 148. The internal programming for the main data collection station 130 can operate such that the patients are placed in a sequential top-to-bottom order based upon whether or not the patient can be generating an alarm signal for one of the patient data being monitored. For example, if one of the patients monitored by monitoring system 130 has a blood pressure exceeding a predetermined maximum amount, this patient can be moved toward the top of the list of patients and the patient's name and/or patient data can be highlighted such that the medical personnel can quickly identify those patients who may be in need of medical assistance. By way of illustration, and without limitation, the following paragraphs is a representative order ranking method for determining the order which the monitored patients are displayed:

Alarm Display Order Patient Status Patients are then sorted 1 Medical Alarm Most alarms violated to least alarms violated, then oldest to newest 2 Missing Data Alarm Oldest to newest 3 Late Oldest to newest 4 Reviewed Medical Alarms Oldest to newest 5 Reviewed Missing Data Oldest to newest Alarms 6 Reviewed Null Oldest to newest 7 NDR Oldest to newest 8 Reviewed NDR Oldest to newest.

Alarm Display Order Patient Status Patients can then sorted 1 Medical Alarm Most alarms violated to least alarms violated, then oldest to newest 2 Missing Data Alarm Oldest to newest 3 Late Oldest to newest 4 Reviewed Medical Alarms Oldest to newest 5 Reviewed Missing Data Oldest to newest Alarms 6 Reviewed Null Oldest to newest 7 NDR Oldest to newest 8 Reviewed NDR Oldest to newest.

As listed in the above, the order of patients listed on the display can be ranked based upon the seriousness and number of alarms that are registered based upon the latest patient data information. For example, if the blood pressure of a single patient exceeds the tolerance level and the patient's heart rate also exceeds the maximum level, this patient will be placed above a patient who only has one alarm condition. In this manner, the medical provider can quickly determine which patient most urgently needs medical attention by simply identifying the patient's name at the top of the patient list. The order which the patients are displayed can be configurable by the remote monitoring system 18 depending on various preferences.

As discussed previously, the escalation server 150 automatically generates a notification message to a specified medical provider for unacknowledged data packets based on user specified parameters.

In addition to displaying the current patient data for the numerous patients being monitored, the software of the main data collection station 130 allows the medical provider to trend the patient data over a number of prior measurements in order to monitor the progress of a particular patient. In addition, the software allows the medical provider to determine whether or not a patient has been successful in recording their patient data as well as monitor the questions being asked by the remote monitoring unit 22.

As previously mentioned, the system 10 uses an intelligent combination of sensors to enhance detection and prediction capabilities. Electrocardiogram circuitry can be coupled to the sensors 14, or electrodes, to measure an electrocardiogram signal of the patient. An accelerometer can be mechanically coupled, for example adhered or affixed, to the sensors 14, adherent patch and the like, to generate an accelerometer signal in response to at least one of an activity or a position of the patient. The accelerometer signals improve patient diagnosis, and can be especially useful when used with other signals, such as electrocardiogram signals and impedance signals, including but not limited to, hydration respiration, and the like. Mechanically coupling the accelerometer to the sensors 14, electrodes, for measuring impedance, hydration and the like can improve the quality and/or usefulness of the impedance and/or electrocardiogram signals. By way of illustration, and without limitation, mechanical coupling of the accelerometer to the sensors 14, electrodes, and to the skin of the patient can improve the reliability, quality and/or accuracy of the accelerometer measurements, as the sensor 14, electrode, signals can indicate the quality of mechanical coupling of the patch to the patient so as to indicate that the device is connected to the patient and that the accelerometer signals are valid. Other examples of sensor interaction include but are not limited to, (i) orthopnea measurement where the breathing rate is correlated with posture during sleep, and detection of orthopnea, (ii) a blended activity sensor using the respiratory rate to exclude high activity levels caused by vibration (e.g. driving on a bumpy road) rather than exercise or extreme physical activity, (iii) sharing common power, logic and memory for sensors, electrodes, and the like.

The signals from the plurality of sensors can be combined in many ways. In some embodiments, the signals may be used simultaneously to determine an impending cardiac decompensation.

In some embodiments, the signals can be combined by using the at least two of the electrocardiogram signal, the respiration signal or the activity signal to look up a value in a previously existing array.

TABLE 1
Lookup Table for ECG and Respiration Signals.

| Heart Rate/Respiration | A-B bpm | C-D bpm | E-F bpm |
|---|---|---|---|
| U-V per min | N | N | Y |
| W-X per min | N | Y | Y |
| Y-Z per min | Y | Y | Y |

Table 1 shows combination of the electrocardiogram signal with the respiration signal to look up a value in a pre-existing array. For example, at a heart rate in the range from A to B bpm and a respiration rate in the range from U to V per minute triggers a response of N. In some embodiments, the values in the table may comprise a tier or level of the response, for example four tiers. In specific embodiments, the values of the look up table can be determined in response to empirical data measured for a patient population of at least about 100 patients, for example measurements on about 1000 to 10,000 patients. The look up table shown in Table 1 illustrates the use of a look up table according to one embodiment, and one will recognize that many variables can be combined with a look up table.

In some embodiments, the table may comprise a three or more dimensional look up table, and the look up table may comprises a tier, or level, of the response, for example an alarm.

In some embodiments, the signals may be combined with at least one of adding, subtracting, multiplying, scaling or dividing the at least two of the electrocardiogram signal, the respiration signal or the activity signal. In specific embodiments, the measurement signals can be combined with positive and or negative coefficients determined in response to empirical data measured for a patient population of at least about 100 patients, for example data on about 1000 to 10,000 patients.

In some embodiments, a weighted combination may combine at least two measurement signals to generate an output value according to a formula of the general form

OUTPUT=aX+bY

where a and b comprise positive or negative coefficients determined from empirical data and X, and Z comprise measured signals for the patient, for example at least two of the electrocardiogram signal, the respiration signal or the activity signal. While two coefficients and two variables are shown, the data may be combined with multiplication and/or division. One or more of the variables may be the inverse of a measured variable.

In some embodiments, the ECG signal comprises a heart rate signal that can be divided by the activity signal. Work in relation to embodiments of the present invention suggest that an increase in heart rate with a decrease in activity can indicate an impending decompensation. The signals can be combined to generate an output value with an equation of the general form

OUTPUT=aX/Y+bZ

where X comprise a heart rate signal, Y comprises an activity signal and Z comprises a respiration signal, with each of the coefficients determined in response to empirical data as described above.

In some embodiments, the data may be combined with a tiered combination. While many tiered combinations can be used a tiered combination with three measurement signals can be expressed as

OUTPUT=(ΔX)+(ΔY)+(ΔZ)

where (ΔX), (ΔY), (ΔZ) may comprise change in heart rate signal from baseline, change in respiration signal from baseline and change in activity signal from baseline, and each may have a value of zero or one, based on the values of the signals. For example if the heart rate increase by 10%, (ΔX) can be assigned a value of 1. If respiration increases by 5%, (ΔY) can be assigned a value of 1. If activity decreases below 10% of a baseline value (ΔZ) can be assigned a value of 1. When the output signal is three, a flag may be set to trigger an alarm.

In some embodiments, the data may be combined with a logic gated combination. While many logic gated combinations can be used, a logic gated combination with three measurement signals can be expressed as

OUTPUT=(ΔX) AND (ΔY) AND (ΔZ)

where (ΔX), (ΔY), (ΔZ) may comprise change in heart rate signal from baseline, change in respiration signal from baseline and change in activity signal from baseline, and each may have a value of zero or one, based on the values of the signals. For example if the heart rate increase by 10%, (ΔX) can be assigned a value of 1. If respiration increases by 5%, (ΔY) can be assigned a value of 1. If activity decreases below 10% of a baseline value (ΔZ) can be assigned a value of 1. When each of (ΔX), (ΔY), (ΔZ) is one, the output signal is one, and a flag may be set to trigger an alarm. If any one of (ΔX), (ΔY) or (ΔZ) is zero, the output signal is zero and a flag may be set so as not to trigger an alarm. While a specific example with AND gates has been shown the data can be combined in may ways with known gates for example NAND, NOR, OR, NOT, XOR, XNOR gates. In some embodiments, the gated logic may be embodied in a truth table.

While the exemplary embodiments have been described in some detail, by way of example and for clarity of understanding, those of skill in the art will recognize that a variety of modifications, adaptations, and changes may be employed. Hence, the scope of the present invention should be limited solely by the appended claims.

What is claimed is:

1. A system for detecting at least one physiological event of a patient, comprising:
a patient measuring system comprising,
an adherent device configured to couple to the patient, the adherent device comprises a plurality of sensors to measure patient data to detect for detecting the at least one physiological event of the patient, and
a wireless communication device coupled to the plurality of sensors and configured to transmit wirelessly patient data;
a remote monitoring system coupled to the wireless communication device and configured to receive the patient data; and
circuitry configured to receive the patient data to detect the physiological event of the patient.

2. The system of claim 1, wherein the remote monitoring system is configured to contact the patient to determine at least one of a patient feeling or a patient thought in response to the circuitry the physiologic event of the patient.

3. The system of claim 2, wherein the remote site is configured to combine the at least one of the patient feeling or the patient thought with the patient data from the adherent device and send a notification in response to the patient data combined with the at least one of the patient feeling or the patient thought.

4. The system of claim 1, wherein the plurality of sensors comprises a combination of sensors to measure at least two of an electrocardiogram signal, a hydration signal, an accelerometer signal or a respiration signal of the patient.

5. The system of claim 1, wherein the patient measuring system further comprises an external device coupled to patient to measure patient data in combination with the adherent device.

6. The system of claim 3, wherein the external device comprises at least one of a weight scale, a blood pressure cuff, a medical treatment device or a medicament dispenser.

7. The system of claim 3, further comprising a monitoring unit configured to detect the at least one physiological event of the patient.

8. The system of claim 5 wherein at least one of the patient measuring system, the remote monitoring system or the monitoring unit comprises the circuitry.

9. The system of claim 5, wherein the patient measuring system comprises monitoring unit.

10. The system of claim 5, wherein the remote monitoring system comprises the monitoring unit.

11. The system of claim 5, wherein each of the external device and the adherent device is coupled to the monitoring unit.

12. The system of claim 5, wherein the external device is configured to communication directly with the adherent device comprising the plurality of sensors and wherein the adherent device is configured to communicate with the monitoring unit.

13. The system of claim 5, wherein the external device and the adherent device are each configured to communicate directly with the monitoring unit.

14. The system of claim 1, further comprising:
a notification device coupled to at least one of the patient measuring system or the remote monitoring system, the notification device configured to provide notification when at least one value received from the plurality of sensors is outside an acceptable physiological range.

15. The system of claim 11, wherein the patient measuring system comprises a monitoring unit and the monitoring unit comprises the notification device and wherein the notification device is located at the monitoring unit.

16. The system of claim 11, wherein the patient measuring system comprises the notification device.

17. The system of claim 11, wherein the remote monitoring system comprises the notification device.

18. The system of claim 11, wherein the notification device is comprises at least one of a visible patient indicator, an audible alarm, an emergency medical service notification, a call center alert, or direct medical provider notification.

19. The system of claim 11, wherein the notification device is configured to provide notification to at least one of, the patient, a clinician, the remote monitoring system, a spouse, a family member, a caregiver or a medical provider and configured to transmit the notification from the notification device to another device.

20. The system of claim 15, wherein notification device is configured to notify two or more of the patient, a clinician, the remote monitoring system, a spouse, a family member, a caregiver or a medical provider, according to a preset hierarchy.

21. The system of claim 16, wherein the preset hierarchy comprises patient notification first and medical provider second.

22. The system of claim 16, wherein the preset hierarchy comprises patient notification second and medical provider first.

23. The system of claim 11, wherein upon receipt of a notification, at least one of a medical provider, the remote monitoring system or a medical treatment device can trigger a high-rate sampling of physiological parameters for alert verification.

24. The system of claim 19, wherein when the values received from the plurality of sensors are not within acceptable physiological ranges the notifying system communicates with the at least one of, the patient, a clinician, a spouse, a family member, a caregiver or a medical provider and from the notification device to another device to allow for therapeutic intervention to prevent decompensation.

25. The system of claim 1, further comprising a processor system and wherein the processor system is configured to store a predetermined target value of the physiological parameters and a series of questions.

26. The system of claim 25, wherein the processor system is configured to compare a sensor measured physiological parameter with the predetermined target value to determine a variance.

27. The system of claim 25, wherein the processor system comprises a processor of the patient detecting system and a processor of the remote monitoring system and wherein the processor of the patient detecting system is configured to accept and store a new predetermined target value and a new series of questions from the processor of the remote monitoring system.

28. The system of claim 1, wherein the first sensor comprises an edema sensor device configured to measure edema of an extremity.

29. The system of claim 1, wherein the patient detecting system includes at least one of a keyboard, a plurality of buttons or a microphone.

30. A method of monitoring a patient to detect at least one physiological event, the method comprising:
adhering an adherent device to a patient to couple the device to the patient, the adherent device comprising a plurality of sensors for measuring patient data to detect the at least one physiological event of the patient;
transferring the patient data directly or indirectly from the plurality of sensors to a remote monitoring system with a wireless communication device coupled to the plurality of sensors; and
detecting the physiological event of the patient at the remote location with the remote monitoring system in response to the patient data.
